(19) United States
(12) Patent Application Publication
(10) Pub. No.: US 2008/0199434 A1
(43) Pub. Date: Aug. 21, 2008

(54) COMPOSITIONS AND METHODS FOR THE TREATMENT OF CARDIOVASCULAR CONDITIONS

(75) Inventors: Jawahar L. Mehta, Little Rock, AR (US); Paul L. Hermonat, Little Rock, AR (US); Jiawei Chen, Shanghai (CN); Chang-Ping Hu, Little Rock, AR (US); Abhijit Dandapat, Galveston, TX (US); Yong Liu, Little Rock, AR (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: BOARD OF TRUSTEES OF THE UNIVERSITY OF ARKANSAS, Little Rock, AR (US)

(21) Appl. No.: 11/938,992

(22) Filed: Nov. 13, 2007

Related U.S. Application Data
(60) Provisional application No. 60/865,565, filed on Nov. 13, 2006.

(51) Int. Cl. A61K 48/00; A61P 9/10; C12Q 1/68; G01N 33/53; A61K 31/711; A61K 31/7105

(52) U.S. Cl. 424/93.2; 514/44; 435/6; 435/7.92

(57) ABSTRACT

The present invention provides methods and compositions for treating a cardiovascular condition. In particular, provided is a method comprising administering to a subject an agent that increases the level and/or activity of angiotensin II type 2 receptors. Also provided is a method for evaluating the risk of having or developing a cardiovascular condition

### CROSS-REFERENCE TO RELATED APPLICATIONS

This application claims priority to U.S. Provisional Application Ser. No. 60/865,565 filed on Nov. 13, 2006, which is hereby incorporated by reference in its entirety.

### FIELD OF THE INVENTION

This invention relates to methods and compositions for diagnosing and/or treating cardiovascular conditions.

### BACKGROUND OF THE INVENTION

Atherosclerosis is a chronic inflammatory response in the walls of arteries due, in large part, to the deposition of lipoproteins, which leads to the formation of multiple plaques within the arteries. Atherosclerosis typically begins in early adolescence, and is usually found in most major arteries, yet it is asymptomatic and not detected by most diagnostic methods. It most commonly becomes seriously symptomatic when interfering with the coronary circulation supplying the heart or cerebral circulation supplying the brain, and is considered the most important underlying cause of heart attacks, strokes, and most cardiovascular conditions. According to United States data for the year 2004, the first symptom of atherosclerotic cardiovascular disease for about 65% of men and 47% of women is a heart attack or sudden cardiac death (death within one hour of onset of the symptom).

Current methods of diagnosing atherosclerosis include cardiac stress testing, the most commonly performed non-invasive method for detecting blood flow limitations. Typically, a cardiac stress test is able to detect only lumen narrowing of ˜75% or greater. Angiography, which may detect lower levels of lumen narrowing, is not only more invasive, but is also limited to those arteries that are examined. Furthermore, recent clinical trials have shown that most severe events occur in locations with heavy plaque but with little or no lumen narrowing.

Other indicators of cardiovascular conditions due to atherosclerosis include high serum levels of LDL, low serum levels of HDL, high serum levels of homocysteine, obesity, diabetes, high blood pressure, and tobacco smoking. If atherosclerosis leads to symptoms, some of symptoms such as angina pectoralis can be treated with pharmaceuticals and/or lifestyle changes. Many individuals at risk for atherosclerosis-related diseases are treated prophylactically with low-dose aspirin and a statin, and are encouraged to lose weight, increase physical activity, and/or stop smoking.

Because of the prevalence of atherosclerosis, there is a need for a method for treating atherosclerosis before it advances to a serious cardiovascular condition. Also needed is a non-invasive method for diagnosing the progression of atherosclerosis and/or evaluating the risk of an individual for a cardiovascular condition due to atherosclerosis.

### SUMMARY OF THE INVENTION

Among the various aspects of the invention, therefore, is an aspect encompassing a method for treating a cardiovascular condition in a subject. The method comprises administering to the subject at least one agent that increases the level and/or activity of an angiotensin II type 2 receptor.

Another aspect of the invention provides a method for evaluating the risk of a subject for a cardiovascular condition. The method comprises determining the ratio of angiotensin II type 2 receptor expression to angiotensin II type 1 receptor expression in a sample from the subject. The risk of having or developing a cardiovascular condition decreases as the ratio increases.

A further aspect of the invention provides a combination of angiotensin II receptor regulators. The combination comprises 1) an agent that increases the level and/or activity of an angiotensin II type 2 receptor and 2) an agent that decreases the level and/or activity of an angiotensin II type 1 receptor or 3) an angiotensin-converting enzyme (ACE) inhibitor.

Other features and aspects of the invention will be in part apparent and in part pointed out hereinafter.

### DESCRIPTION OF THE FIGURES

This application file contains at least one drawing executed in color. Copies of this patent application publication with color drawings will be provided by the U.S. Patent and Trademark Office upon request and payment of the necessary fee.

FIG. 1 illustrates that angiotensin II type 2 receptor (AT2R) was over-expressed in LDL receptor knockout (LDLR KO) mice administered AAV/AT2R. Panel (A) illustrates that AT2R cDNA vector was amplified in DNA isolated from LDLR KO mice given AAV/AT2R. Panel (B) presents an image of a PCR gel and a plot of the relative levels of AT2R mRNA in each treatment group. Panel (C) presents images of Western blots and a plot of the relative levels of AT2R protein in each treatment group (n=5 per group).

FIG. 2 illustrates that angiotensin II type 1 receptor (AT1R) was expressed at similar levels in all KO mice. Panel (A) presents an image of a PCR gel and a plot of the relative levels of AT1R mRNA in each treatment group. Panel (B) presents images of Western blots and a plot of the relative levels of AT1R protein in each treatment group (n=5 per group).

FIG. 3 illustrates the role of AT2R over-expression in atherosclerotic lesion formation. Panel (A) presents an image (top) showing representative aortas from each group and a plot (bottom) of the average percent of sudanophilic areas of the aortas (n=5-7 per group). Panel (B) present images (top) of representative aortas and a plot (bottom) of the average intima thickness in each group (n=5-7 per group). Arrows indicate regions of intimal thickening. Panel (C) presents images of cross-sectional areas of aorta stained for α-actin to reveal vascular smooth muscle cells (SMCs). The inset illustrates the presence of SMCs in the proliferating intima.

FIG. 4 illustrates the modulation of downstream regulators of atherogenesis by AT2R over-expression. Panel (A) presents images of Western blots and a plot of the relative levels of NADPH oxidase (p47^{phox} and p22^{phox} subunits) in each treatment group. Panel (B) presents images of Western blots and a plot of the relative levels of NF-κB (p65) in each group. Panel (C) presents images of Western blots and a plot of the relative levels of nitrotyrosine in each group. Panel (D) presents images of aortas stained for nitrotyrosine. Data are from 3-5 mice per group.

FIG. 5 illustrates the modulation of additional downstream regulators of atherogenesis by AT2R over-expression. Panel (A) presents images of Western blots and a plot of the relative levels of Akt-1 and phosphorylated Akt-1 in each group. Panel (B) presents images of Western blots and a plot of the relative levels of eNOS and phosphorylated-S1177 eNOS in each group. Panel (C) presents images of Western blots and a plot of the relative levels of NO-1 in each group. Panel (D) presents images of Western blots and a plot of the relative levels of LOX-1 in each group. Data are from 3-5 mice per group.

FIG. 6 illustrates collagen deposition and the expression of pro-collagen I, osteopontin, and fibronectin. Panel (A) presents images of aortas stained with dyes to visualize collagen (left) and immunostained for osteopontin or fibronectin (right) for each treatment group. Panel (B) presents of the average area of collagen, as revealed by Masson's trichrome stain, for each group. Panel (C) presents images of Western blots for the indicated proteins.

FIG. 7 presents matrix metalloproteinase (MMP) expression and activity. Panel (A) presents images of Western blots and a plot of the relative levels of MMP-9 and MMP-2 in each treatment group. Panel (B) presents images of zymograms and a plot of the activity of MMP-9 and MMP-2 in each treatment group.

FIG. 8 illustrates the levels of expression and/or activity of oxidant signaling proteins. Panel (A) presents a plot of Cu/Zn SOD activity in each treatment group. Panel (B) presents images of Western blots and a plot of the relative levels of CD-68 in each group. Panel (C) presents images of Western blots and a plot of the relative levels of p38 MAPK and phosphorylated p38 MAPK in each group. Panel (D) presents images of Western blots and a plot of the relative levels of p44/42 MAPK and phosphorylated p44/42 MAPK in each group.

### DETAILED DESCRIPTION OF THE INVENTION

It has been discovered that increasing the levels and/or activity of the angiotensin II type 2 receptor (AT2R) reduces the formation of atherosclerotic plaques. Upregulation of AT2R appears to counteract and mitigate the negative effects of the angiotensin II type 1 receptor (AT1R) in atherogenesis. Thus, agents that increase the level and/or activity of AT2R may be used to treat the multitude of cardiovascular conditions associated with atherosclerosis. Furthermore, the relative levels of AT2R and AT1R may be used as a diagnostic indicator of the risk for a cardiovascular condition, wherein a high ratio of AT2R to AT1R indicates a low risk of having or developing a cardiovascular condition. This invention, therefore, provides a non-invasive method for diagnosing atherosclerosis and its ensuing cardiovascular conditions, as well as methods and compositions for treating atherosclerosis before it progresses into a serious cardiovascular condition.

### I. Method for Treating a Cardiovascular Condition

One aspect of the present invention encompasses a method for treating a cardiovascular condition in a subject. The method comprises administering to the subject at least one agent that increases the level and/or activity of AT2R.

a. Cardiovascular Conditions

In general, the cardiovascular condition includes, but is not limited to, hypertension (including essential hypertension, pulmonary hypertension, pulmonary arterial hypertension, secondary hypertension, isolated systolic hypertension, hypertension associated with diabetes, hypertension associated with atherosclerosis, and renovascular hypertension); complications associated with hypertension (including vascular organ damage, congestive heart failure, angina, stroke, glaucoma and impaired renal function); valvular insufficiency; stable, unstable and variant (Prinzmetal) angina; peripheral vascular disease; myocardial infarction; stroke (including stroke recovery); thromboembolic disease; restenosis; arteriosclerosis; atherosclerosis; angiostenosis after bypass; angioplasty (including percutaneous transluminal angioplasty and percutaneous transluminal coronary angioplasty); hyperlipidemia; hypoxic vasoconstriction; vasculitis (including Kawasaki's syndrome); heart failure (including congestive heart failure, decompensated heart failure, systolic heart failure, diastolic heart failure, left ventricular heart failure, right ventricular heart failure, and left ventricular hypertrophy); ischemia (including coronary ischemia, myocardial ischemia, renal ischemia, cerebrovascular ischemia); Raynaud's phenomenon; preeclampsia; pregnancy-induced high blood pressure; cardiomyopathy; and arterial occlusive disorders. In preferred embodiments, the cardiovascular condition may be atherosclerosis, coronary occlusion, myocardial infarction, renal ischemia, cerebrovascular ischemia, ischemic/reperfusion injury, hypertension, arterial aneurysm, and peripheral vascular disease.

b. Agent

The agent that increases the level of AT2R and/or increases the activity of AT2R may be a nucleic acid encoding an AT2R polypeptide. In some embodiments, the nucleic acid encoding an AT2R polypeptide may be an RNA molecule. In other embodiments, the nucleic acid encoding an AT2R polypeptide may be a DNA molecule, such as genomic DNA (i.e., exonic and intronic sequences) or cDNA. In preferred embodiments, the nucleic acid encoding an AT2R polypeptide may be a cDNA sequence, such as SEQ ID NO. 1 or SEQ ID NO. 3. One of skill will recognize that, because of codon degeneracy, a number of nucleic acid sequences may encode the same polypeptide. Accordingly, the nucleic acid may be “substantially identical” to SEQ ID NO:1, for example.

Two nucleic acid sequences are said to be “identical” if the sequence of nucleotides in the two sequences is the same when aligned for maximum correspondence. Optimal alignment of sequences for comparison may be conducted by using the BLASTn algorithm of Altschul et al. (J. Mol. Biol. 215:403-410, 1990). This algorithm is incorporated into the BLAST programs at the NCBI website (http://www.ncbi.nlm.nih.gov/BLAST/). To obtain gapped alignments for comparison purposes, Gapped BLAST may be utilized as described in Altschul et al. (Nucleic Acids Res. 25:3389-3402, 1997). When utilizing BLAST and Gapped BLAST programs, the default parameters of the respective programs are employed. The percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which an identical nucleotide occurs in both sequences to yield the number of matched positions, dividing the number of matched positions by the total number of positions in the window of comparison and multiplying the result by 100 to yield the percentage of sequence identity. The term “substantial identity” of nucleic acids means that a nucleic acid comprises a sequence that has at least 60% sequence identity, preferably at least 80%, more preferably at least 90% and most preferably at least 95%, compared to a reference sequence.

In other embodiments, the nucleic acid encoding an AT2R polypeptide may be operably linked to at least one expression control sequence. Expression control sequences include transcriptional control elements such as promoters, enhancers, polyadenylation sites, termination sequences, and splice sites, as well as translational control elements such Kozak sequences, ribosome binding sites, 5′ UTR regulatory sequences, and 3′ UTR regulatory sequences. In one embodiment, the nucleic acid encoding an AT2R polypeptide may be an RNA sequence operably linked to at least one expression control element.

In another embodiment, the nucleic acid encoding an AT2R polypeptide may be a DNA sequence operably linked to at least one transcriptional control element, such as a promoter. In preferred embodiments, the promoter may be a cardio-specific, a vascular smooth muscle cell-specific promoter, or a fibroblast-specific promoter. Accordingly, suitable promoters include, but are not limited to, an oxidized low density lipoprotein (lectin-like) receptor 1 (LOX-1) promoter, a cardio-specific alpha-myosin heavy chain (αMHC) promoter (Palermo et al., 1995, Cell Mol. Biol. Res. 41:501-519), a cardio-specific alpha-actin promoter (Sartorelli et al., 1990, Genes Dev. 4: 1811-1822), a myosin light chain-2v (MLC2v) promoter (Lee et al., Mol. 1994, Cell Biol. 14:1220-1229); a SM22 promoter specific for vascular smooth muscle cells (Kallmeier et al. 1995, J. Biol. Chem. 270:30949-30957), a MusA promoter (i.e., a synthetic promoter specific for cardiomyocytes), a MusB promoter (i.e., a synthetic promoter specific for aortic smooth muscle cells), a fibroblast specific protein 1 (FSP1) promoter (Okada et al., 1998, Am. J. Physiol.-Renal Physiol. 275:F306-F314), and a pro-α2(I) collagen (collα2) promoter (Denton et al., 2005, J. Biol. Chem. 280(18):16053-16065).

In other embodiments, the nucleic acid encoding an AT2R polypeptide may further comprise a vector. In general, administration of a vector carrying the nucleic acid encoding an AT2R polypeptide may ensure sustained and long-term expression of the AT2R polypeptide. The vector may be an adenovirus vector, a lentivirus vector, a retrovirus vector, or an adeno-associated virus (AAV) vector. Non-limiting examples of suitable adenovirus vectors include those based on the human adenoviruses A, B, C, D, E, and F. Suitable lentivirus vectors include those based on human immunodeficiency virus 1 (HIV), simian immunodeficiency virus (SIV), and feline immunodeficiency virus (FIV). Examples of suitable retrovirus vectors include those based on A-type, B-type, C-type, or D-type viruses. Suitable adeno-associated viruses include AAV type 1, AAV type 2, AAV type 3, AAV type 4, AAV type 5, AAV type 6, AAV type 7, AAV type 8, AAV type 9, AAV type 10, and AAV type 11. In preferred embodiments, the vector may be an AAV type 2 based vector, an AAV type 6 based vector, an AAV type 8 based vector, or an AAV type 9 based vector. Those of skill in the art will appreciate that the vector may comprise additional elements, such as at least one selectable marker gene. In other embodiments, rather than being part of vector, the nucleic acid encoding an AT2R polypeptide may be delivered using other means that are well known in the art. For example, a naked nucleic acid molecule may be delivered via microinjection into cells or cell nuclei; a nucleic acid coated metallic particle may be delivered via a gene gun; a nucleic acid liposome construct may be delivered via lipofection; or a nucleic acid may be delivered using a nanotube or nanostructure delivery system.

A nucleic acid encoding an AT2R polypeptide may be obtained using standard molecular biology procedures well known to those with skill in the art. Guidance may be found in Current Protocols in Molecular Biology (Ausubel et al., John Wiley & Sons, New York, 2003) or Molecular Cloning: A Laboratory Manual (Sambrook & Russell, Cold Spring Harbor Press, Cold Spring Harbor, N.Y., 3^{rd} edition, 2001). In some embodiments, a nucleic acid encoding an AT2R polypeptide may be obtained via an amplification technique, such as the polymerase chain reaction (PCR). For example, a genomic DNA sequence encoding an AT2R polypeptide may be obtained via PCR or long distance PCR. Alternatively, a complete cDNA sequence encoding an AT2R polypeptide may be obtained by reverse transcriptase-PCR or rapid amplification of cDNA ends (RACE), e.g., 5′ RACE or 3′ RACE. In other embodiments, the nucleic acid sequence encoding an AT2R polypeptide may be obtained via screening a genomic or a cDNA library under stringent conditions using techniques well known in the art. The nucleic acid encoding an AT2R polypeptide may be cloned, inserted into a vector, and/or operably linked to appropriate expression control elements using standard procedures.

In other embodiments, the agent that increases the level of AT2R and/or increases the activity of AT2R may be an AT2R polypeptide. Non-limiting examples of suitable AT2R polypeptides include SEQ ID NO:2 and SEQ ID NO:4. One of skill will recognize that polypeptides may be “substantially similar” in that an amino acid may be substituted with a similar amino acid residue without affecting the function of the mature protein. The degree of sequence identity between two amino acid sequences may be determined using the BLASTP algorithm of Karlin and Altschul (Proc. Natl. Acad. Sci. USA 87:2264-2268, 1993), essentially as detailed above. Substantial identity of amino acid sequences for these purposes normally means sequence identity of at least 35%, preferably at least 60%, more preferably at least 90%, and most preferably at least 95%. Polypeptide sequences which are “substantially similar” share sequences as noted above except that residue positions, which are not identical, may have conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acid substitution groups include: valine-leucine-isoleucine, phenylalanine-tyrosine, lysine-arginine, alanine-valine, and asparagine-glutamine.

An AT2R polypeptide may be an isolated naturally occurring polypeptide, a recombinantly produced polypeptide, a synthetically produced polypeptide, or a polypeptide produced by a combination of these methods. Means for preparing such polypeptides are well understood in the art.

In still other embodiments, the agent that increases the activity of AT2R and/or increases the activity of AT2R may be an AT2R agonist or a physiologically acceptable salt thereof. In preferred embodiments, the AT2R agonist may be a selective agonist. Stated another way, the agonist specifically binds and activates AT2R but not AT1R. The selective AT2R agonist may be a peptide, a peptide mimetic, or an organic molecule. In preferred embodiments, the selective AT2R agonist may be a peptide, such as compound CPG 42112A (which is N-α-nicotinoyl-Tyr-(N-α-carbobenzyloxy-Arg)-Lys-His-Pro-Ile-OH; SEQ ID NO:7), p-aminophenylalanine-angiotensin II, angiotensin(1-7) heptapeptide, or a peptidergic agonist disclosed in U.S. Pat. No. 5,834,432 incorporated herein in its entirety by reference.

c. Administration

The agent may be administered to the subject by a variety of routes. Non-limiting examples of suitable routes of administration include oral, inhalation, transdermal, transmucosal (e.g., intranasal or rectal), intravenous, intramuscular, and subcutaneous. Agents administered parenterally, i.e., intravenously, intramuscularly, and subcutaneously, may include a sterile diluent such as water, saline solution, a pharmaceutically acceptable polyol such as glycerol, propylene glycol, polyethylene glycols, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the solution may be adjusted with acids or bases such as hydrochloric acid or sodium hydroxide. Preparations for oral administration generally include an inert diluent or an edible carrier. They may be include a pharmaceutically compatible binding agent such as microcrystalline cellulose, gum tragacanth or gelatin; an excipient such as starch or lactose; a disintegrating agent such as alginic acid, Primogel, or corn starch; a lubricant such as magnesium stearate or sterotes; a glidant such as colloidal silicon dioxide; a sweetening agent such as sucrose or saccharin; and/or a flavoring agent such as peppermint, methyl salicylate, or citrus flavoring. Oral preparations may be enclosed in gelatin capsules, compressed into tablets, or prepared as a fluid carrier. For administration by inhalation, the agent is generally delivered in the form of an aerosol spray from a pressurized container or dispenser that contains a suitable propellant, e.g., a gas such as carbon dioxide, or a nebulizer. For transdermal or transmucosal administration, penetrants appropriate to the barrier to be permeated are generally included in the preparation. Transmucosal administration may be accomplished through the use of nasal sprays or suppositories, and transdermal administration may be via ointments, salves, gels, patches, or creams as generally known in the art.

In a preferred embodiment, the agent comprises an adeno-associated virus vector comprising DNA encoding AT2R that is operably linked to a cardio-specific, a vascular smooth muscle cell-specific promoter, or fibroblast-specific promoter, and the agent is administered intravenously. Generally speaking, the expression of AT2R in cardiac and/or vascular cells is sustained over an extended period of time.

d. Subject

The subject administered the agent that increases the level of AT2R and/or increases the activity of AT2R may vary. Suitable subjects include animals and humans. The animal may be a companion animal such as a cat, a dog or a horse; a research animal such as a mouse, a rat, a rabbit, or a pig; an agricultural animal such as a cow, a pig, a goat, sheep, a chicken, a duck, or a goose; a zoo animal; or a primate such as a chimpanzee, a monkey, or a gorilla. In preferred embodiments, the subject is a human.

### II. Method for Evaluating the Risk for a Cardiovascular Condition

A further aspect of the invention provides a method for evaluating the risk of a subject for a cardiovascular condition. The method comprises determining the ratio of AT2R expression to AT1R expression in a sample from the subject, wherein the risk of having or developing a cardiovascular condition decreases as the ratio increases. In general, the ratio of AT2R to AT1R will be compared to a baseline value. For example, the baseline ratio of AT2R:AT1R in adult endothelial cells is about 1:3 (Li et al., 1999, Am. J. Physiol. 276:H78-92). In one embodiment, the ratio of AT2R to AT1R may be elevated about 1.5-fold relative to a baseline value. In another embodiment, the ratio of AT2R to AT1R may be elevated about 2-fold relative to the baseline value. In still another embodiment, the ratio of AT2R to AT1R may be elevated about 3-fold relative to the baseline value. In an alternate embodiment, the ratio of AT2R to AT1R may be elevated about 4-fold relative to the baseline value. In another alternate embodiment, the ratio of AT2R to AT1R may be elevated about 5-fold relative to the baseline value. In yet another embodiment, the ratio of AT2R to AT1R may be elevated about 10-fold relative to the baseline value. In general, an increased ratio of AT2R to AT1R indicates a decreased risk of having or developing a cardiovascular condition. Cardiovascular conditions were detailed above in section Ia, and subjects were detailed above I section Id.

Measuring the levels of expression of AT2R and AT1R may be accomplished by a variety of techniques, all of which are well known in the art and are detailed in molecular biology reference books such as Ausubel (2003, supra) and Sambrook and Russell (2001, supra). In one embodiment, the levels of AT2R and AT1R polypeptides may be measured quantitatively using an enzyme-linked immunosorbent assay (ELISA). In another embodiment, the relative levels of AT2R and AT1R polypeptides may be determined using Western blotting. In yet another embodiment, the relative levels of AT2R and AT1R polypeptides may be determined via protein dot blotting. In an alternate embodiment, the relative levels of AT2R and AT1R polypeptides may be determined using immunohistochemical or immunoflorescence localization in sections of tissue. In still another embodiment, levels of AT2R and AT1R polypeptides may be measures using a protein microarray or an antibody microarray.

In an alternate embodiment, the levels of AT2R and AT1R mRNAs may be measured using reverse transcriptase PCR or reverse transcriptase quantitative PCR. In another embodiment, the relative levels of AT2R and AT1R mRNAs may be determined using Northern blotting. In still another embodiment, the relative levels of AT2R and AT1R mRNAs may be determined via RNA dot blotting. In yet another embodiment, the relative levels of AT2R and AT1R mRNAs may be determined using a nuclease protection assay. In a further embodiment, the relative levels of AT2R and AT1R mRNAs may be determined via in situ hybridization. In another alternate embodiment, the levels of AT2R and AT1R mRNAs may be measured using a nucleic acid microarray.

The sample in which the levels of AT2R and AT1R expression are measured can and will vary. Suitable samples include a body fluid or a tissue specimen. Non-limiting examples of body fluids include blood, serum, plasma, saliva, cerebrospinal fluid, pleural fluid, lymphatic fluid, milk, sputum, semen, tears, and urine. Suitable tissue specimens include biopsied tissue samples such a vascular tissue sample, an arterial tissue sample, an aorta tissue sample, and a myocardial tissue sample.

Protein or RNA may be isolated from the body fluid or tissue sample using techniques well known in the art and disclosed in standard molecular biology reference books, as listed above. In some embodiments, the tissue sample may be fixed, embedded in plastic or paraffin, and sectioned using standard procedures. In still other embodiments, the tissue sample may be frozen and cryosectioned using techniques well known in the art.

### III. A Combination of Angiotensin II Receptor Regulators

Yet another aspect the invention provides a combination comprising 1) an agent that increases the level and/or activity of AT2R and 2) an agent that decreases the level and/or activity of AT1R or 3) an angiotensin-converting enzyme (ACE) inhibitor.

The agent that increases the level and/or activity of AT2R may be a nucleic acid encoding AT2R, an AT2R polypeptide, or an AT2R agonist, as detailed above in section Ib. The agent that decreases the level and/or activity of AT1R may be an AT1R antisense nucleic acid or an AT1R antagonist. In general, the AT1R antisense nucleic acid interacts with endogenous AT1R mRNA molecules such that AT1R mRNA is cleaved and/or degraded or translation of AT1R is reduced or inhibited. Suitable antisense nucleic acids include oligodeoxynucleotides, small RNA molecules, short interfering RNA molecules, and short hairpin RNA molecules.

The antisense nucleic acid may comprise standard nucleotides (i.e., adenosine, guanosine, cytidine, thymidine, and uridine) or nucleotide analogs. A nucleotide analog refers to a nucleotide having a modified (purine or pyrimidine) base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos. The nucleotides may be linked by phosphodiester, phosphothioate, phosphoramidite, or phosphorodiamidate bonds.

Suitable AT1R antagonists include, but are not limited to, candesartan, eprosartan, irbesartan, losartain, olmesartan, telmisartan, and valsartan. Without being bound by any particular theory, it is believed that AT1R antagonists bind AT1R, leading to elevated levels of plasma angiotensin II, which in turn binds to and activates AT2R.

Non-limiting examples of suitable ACE inhibitors include captopril, benazepril, enalapril, fosinopril, lisinopril, perindopril, quinapril, ramipril, and zofenopril. In general, an ACE inhibitor blocks the conversion of angiotensin I to angiotensin II, thereby leading to reduced plasma levels of angiotensin II.

Routes of administration and formulations were detailed above in section Ic.

### DEFINITIONS

To facilitate understanding of the invention, several terms are define below.

“Angiotensin II type 2 receptor,” also known as AT2R or AGTR2, and “angiotensin II type 1 receptor,” also known as AT1R or AGTR1, are G protein-coupled receptors that are bound and activated by the ligand, angiotensin II.

The term “treating,” as used herein, refers to alleviating, reversing, inhibiting the progress of, and/or preventing a cardiovascular condition. In general, the cardiovascular condition is associated with atherosclerosis. The term “treatment”, as used herein, unless otherwise indicated, refers to the act of treating as “treating” is defined immediately above.

As various changes could be made in the above-described methods and compositions without departing from the scope of the invention, it is intended that all matter contained in the above description and the examples presented below, shall be interpreted as illustrative and not in a limiting sense.

### EXAMPLES

The following examples illustrate various aspects of the invention.

### Examples 1-3

### Over-Expression of Angiotensin II Type 2 Receptor Reduces Atherogenesis and Modulates LOX-1, Endothelial Nitric Oxide Synthase, and Heme-Oxygenase-1 Expression

### Background.

Atherosclerosis, at least in part, is an inflammatory/immune disorder accompanied by oxidative stress. Angiotensin (Ang) II type 1 receptor (AT1R) expression is upregulated in atherosclerosis and plays an important role in atherogenesis. Since angiotensin II type 2 receptor (AT2R) activity is purported to oppose the effects of AT1R, it was hypothesized that AT2R (agtr2) over-expression would inhibit atherogenesis. LOX-1, a lectin-like receptor for oxidized LDL (ox-LDL), is the major receptor responsible for binding, internalization and degradation of ox-LDL in endothelial cells and plays a critical role in atherogenesis. Ang II upregulates LOX-1 expression via AT1R activation. There is also evidence for a reduction in the expression and activity of endothelial nitric oxide synthase (eNOS) in atherosclerosis. Heme-oxygenase-1 (HO-1), an enzyme involved in the genesis of the potent vasodilator species carbon monoxide, is also altered in atherosclerosis. Both eNOS and HO-1 are regulated by Ang II.

These studies were designed to determine 1) whether AT2R cDNA delivered with adeno-associated virus type 2 (AAV) would result in sustained over-expression of AT2R in vascular tissues; 2) whether over-expression of AT2R would inhibit atherogenesis in LDL receptor-deficient (LDLR KO) mice; and 3) whether AT2R over-expression would modulate LOX-1, eNOS and HO-1 expression.

### Experimental Protocols for Examples 1-3.

Construction of AAV/AT2R vector and generation of AAV/AT2R stocks. Mouse AT2R (agtr2) cDNA was generated by PCR amplification. Total RNA was isolated from aortic tissues of C57BL/6 mice using Trizol reagent (Invitrogen, Carlsbad, Calif.) and treated with 2.0 U/μg of RNase-free DNase I (Promega, Madison, Wis.) at 37° C. for 1 hour. mRNA was separated using the Oligotex mRNA Mini Kit (QIAGEN, Valencia, Calif.). First-strand cDNA synthesis was performed using oligo(dT)15 primers. PCR amplification for the AT2R cDNA sequence was carried out using the following primer pair: 5′-GAGTTGCTGCAGTTCAAT-3′ (SEQ ID NO:5) and 5′-GAACTGTATTATACGTATGCCAC-3′ (SEQ ID NO:6) that amplify the sequence from nucleotides 149 to 1326 (NCBI Gene Bank: NM-007429). After AT2R cDNA was sequenced, it was ligated into an AAV vector, dl6-95, as described by Li et al. (2006, Biochem. Biophys. Res. Commun. 344:701-707). Hereafter, recombinant AAV (rAAV) vector is referred to as AAV/AT2R. The rAAV virus stocks were generated as described by Li et al. (2006). The titer of purified virus, in encapsidated genomes (eg) per milliliter (eg/ml), was calculated by dot-blot hybridization and determined to be about 10¹¹ eg/ml.

Animal protocol. Wild-type C57BL/6 mice and homozygous LDLR KO mice (on C57BL/6 background) were obtained from Jackson Laboratories (Bar Harbor, Me.). Group 1: Wild-type mice were injected with 100 μL of saline via the tail vein (negative control group). Group 2: LDLR KO mice were injected with 100 μL of saline via the tail vein (positive control group). Group 3: LDLR KO mice were injected with 100 μL of AAV/AT2R virus (10¹⁰ eg) (experimental group). Group 4: LDLR KO mice were injected with 100 μL of AAV/Neo virus (10¹⁰ eg) (AAV control group). All animals were kept on a high-cholesterol (4% cholesterol/10% cocoa butter) for 18 weeks. All experimental procedures were performed in accordance with the protocols approved by the Institutional Animal Care and Usage Committee at the University of Arkansas for Medical Sciences.

Analysis of atherosclerotic lesions, plasma lipids, and expression patterns. After harvesting the animals, aortic fatty deposits (index of atherosclerotic lesion formation) and intima thickness were quantitated by Sudan IV and H&E staining, respectively (Mehta et al. 2007, Circ. Res. 100:1634-1642; Li et al., 2006). Plasma levels of total cholesterol, HDL-cholesterol, triglycerides, were measured by a clinical laboratory. Total DNA was isolated from the frozen tissue specimens from the AAV or saline injected LDLR KO mice. PCR amplification was performed as described by Li et al. (2006). Aortic tissues were immunohistochemically stained for a-actin and nitrotyrosine as described by Li et al. (2006). Aortic specimens were derived from animals at 18 weeks of high cholesterol diet. The RNA isolation and RT-PCR amplification analysis of AT1R and AT2R were carried out as described by Li et al. 1999, Circ. Res. 84:1043-1049. Protein expression of AT1R, AT2R, NADPH p22^{phox}, NADPH p47^{phox}, NF-κB p65, nitrotyrosine, Akt-1, phos-Akt-1, eNOS, phos-S1177 eNOS, HO-1 and LOX-1 was carried out by Western blotting as described by Mehta et al. (2007). Band density relative to β-actin was analyzed. Data are presented as mean ±SEM. All values were analyzed by using one-way ANOVA and the Newman-Keuls-Student t test. A P value ≦0.05 was considered significant.

### Example 1

### Over-Expression of AT2R in LDLR-Deficient Mice

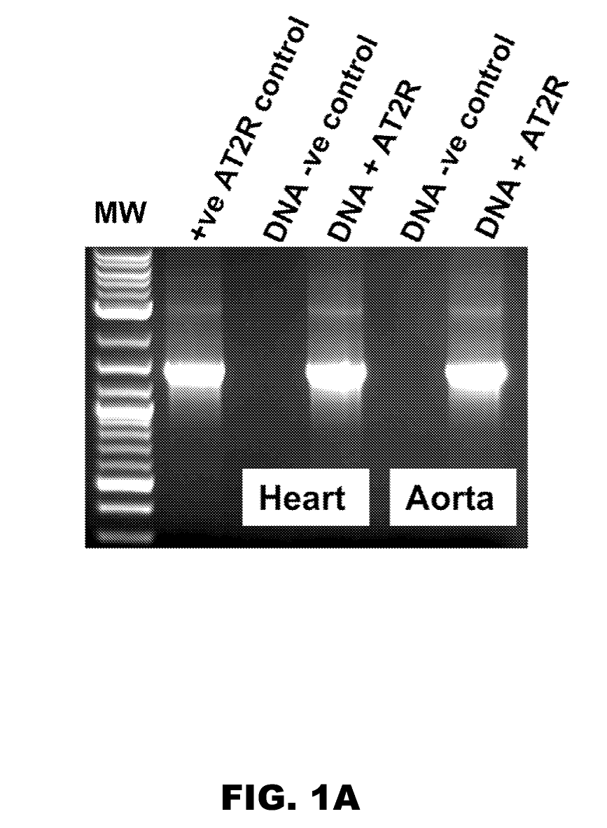

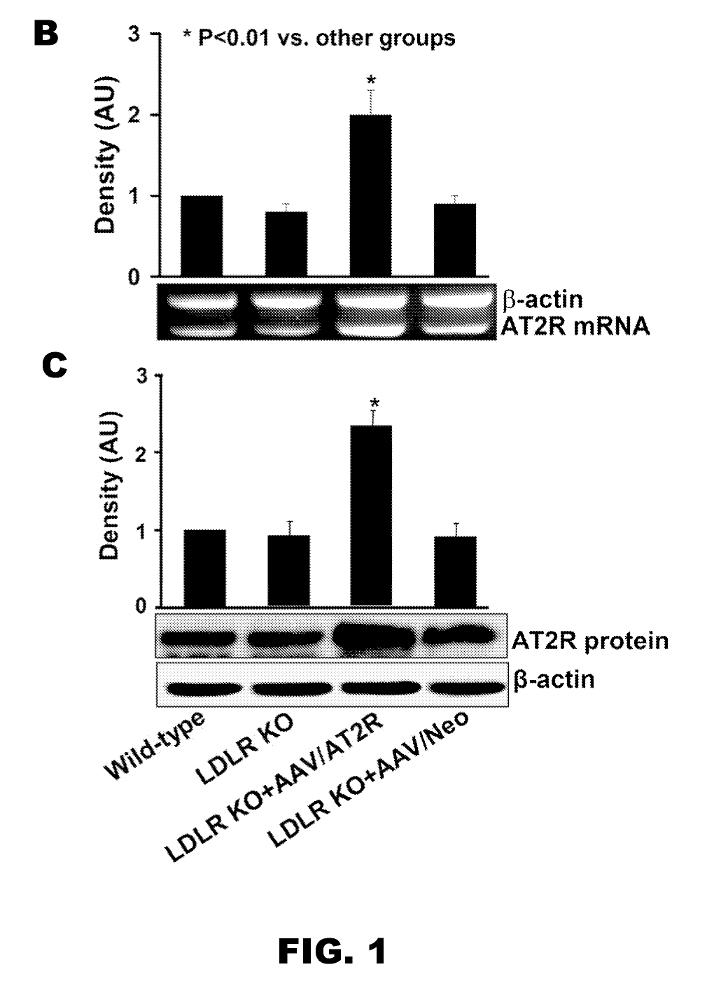

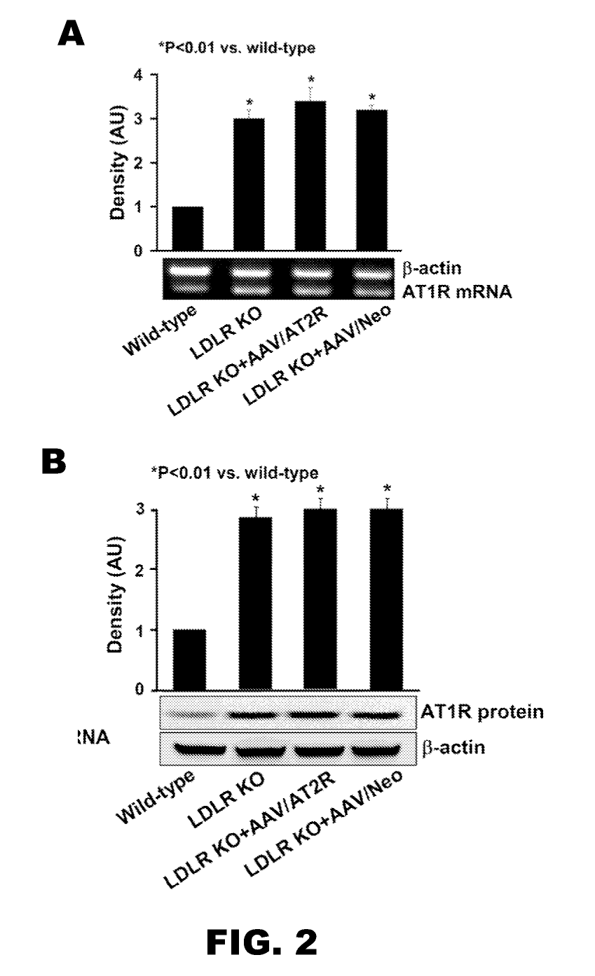

The AT2R vector cDNA was present in cardiovascular tissue (i.e., heart and aorta) of LDLR KO mice 18 weeks after administration of AAV/AT2R and high cholesterol diet feeding (FIG. 1A). There was also a marked increase in AT2R mRNA (FIG. 1B) and protein (FIG. 1C) in mice given AAV/AT2R, as compared to saline-injected or AAV/Neo-injected mice.

The levels of AT1R expression (mRNA and protein) were markedly increased in atherosclerotic aortas from all LDLR KO mice (FIGS. 2A and 2B), as compared to the aortas of wild-type mice. Administration of saline, AAV/AT2R, or AAV/Neo did not affect AT1R expression. All of the LDLR KO mice on a high-cholesterol diet had elevated total and LDL-cholesterol levels in plasma (P<0.01 vs. C57BL/6 mice) (data not shown). Administration of saline, AAV/AT2R, or AAV/Neo, therefore, had no effect on plasma lipid levels.

Thus, administration of a vector comprising AT2R coding sequence via a single tail injection results in increased and sustained levels of AT2R mRNA and protein in vascular tissues.

### Example 2

### Over-Expression of AT2R and Inhibition of Atherosclerotic Lesions

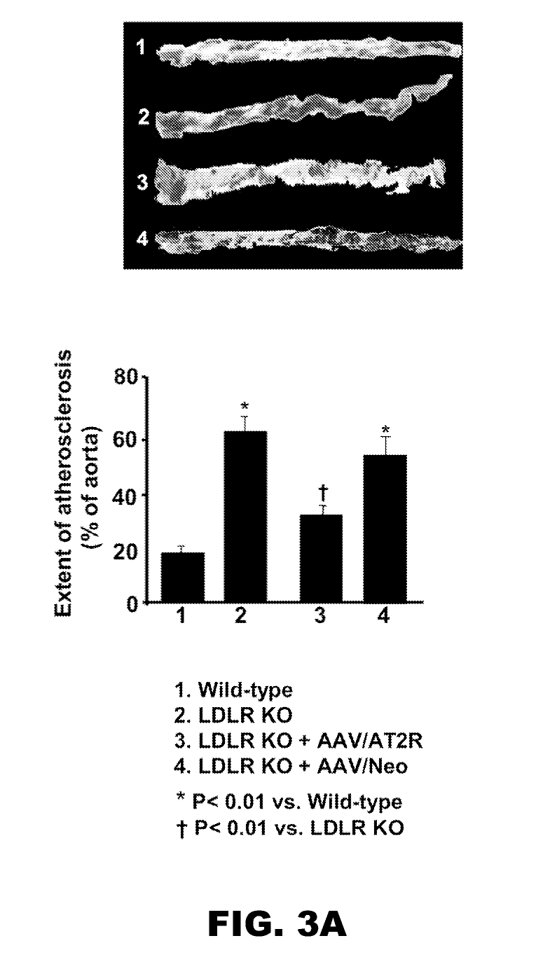

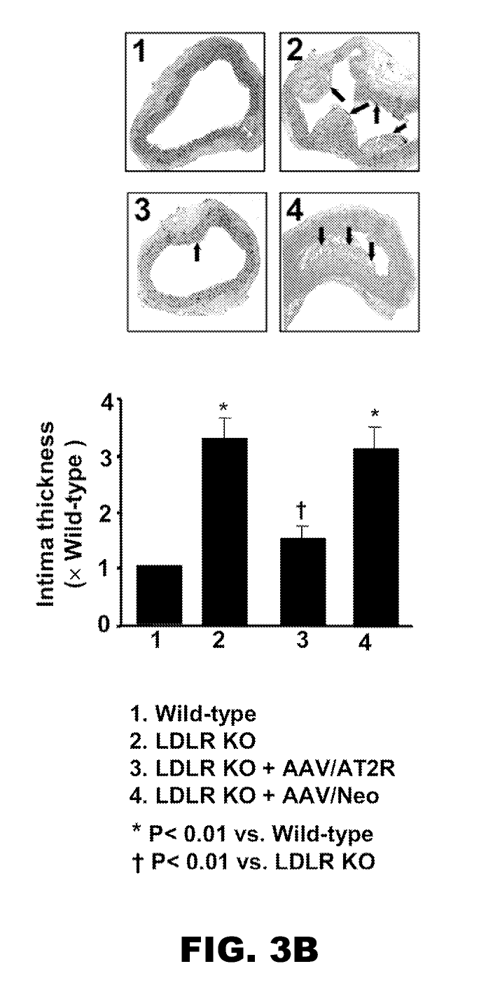

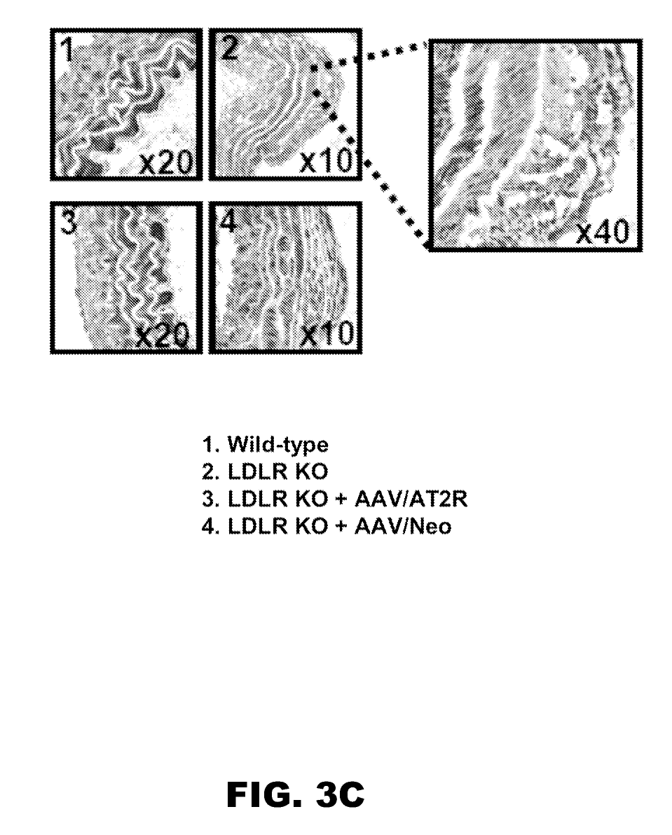

Analysis of the aortas revealed that wild-type mice had some areas of sudanophilia (˜20% of the aorta), which may be an indication of the very high cholesterol diet. Saline-treated and AAV/Neo-treated LDLR KO mice had about 60% of the aorta covered with areas of sudanophilia (P<0.01 vs. wild-type mice). In contrast, AAV/AT2R treated LDLR KO mice displayed dramatically smaller areas of sudanophilia, such that ˜30% of the aorta was sudanophilic (FIG. 3A). The data on the extent of sudanophilic areas were complemented by the data on cross-sections of aorta wherein intimal area was examined. The intima was several-fold thicker in saline-treated and AAV/Neo-treated LDLR KO mice, as compared to wild-type mice, and contained a large number of macrophages. The over-expression of AT2R in LDLR KO mice resulted in a consistent reduction in intimal thickness, as well as a reduction in the number of macrophages (FIG. 3B). Furthermore, there was extensive disarrayed SMC accumulation in the proliferating intima in saline-treated and AAV/Neo-treated LDLR KO mice, characteristic of atherosclerosis. In contrast, AAV/AT2R treated LDLR KO mice did not show excessive SMC accumulation (FIG. 3C).

### Example 3

### Mechanisms of Inhibition of Atherosclerosis by AT2R Over-Expression

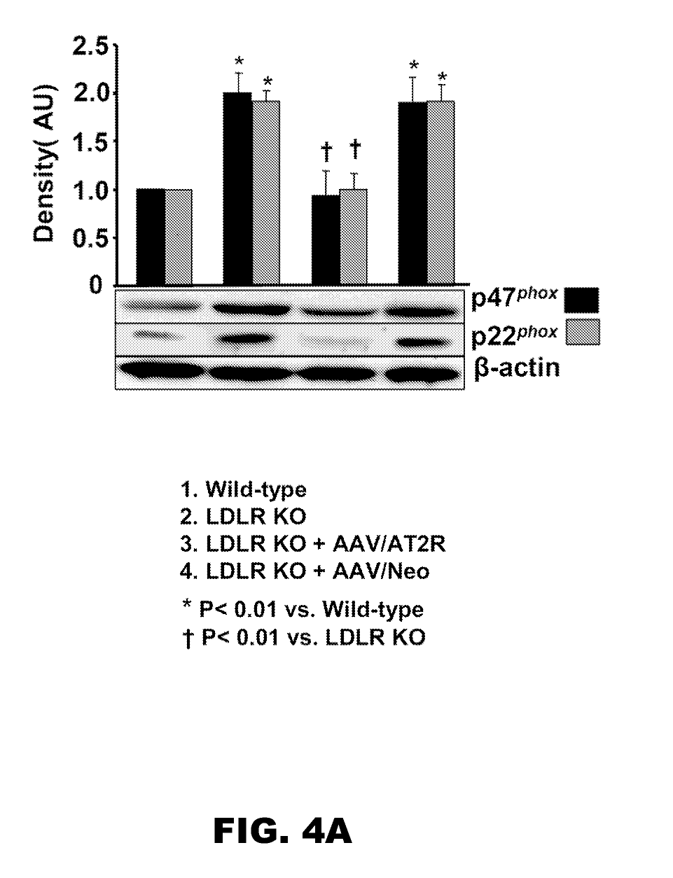

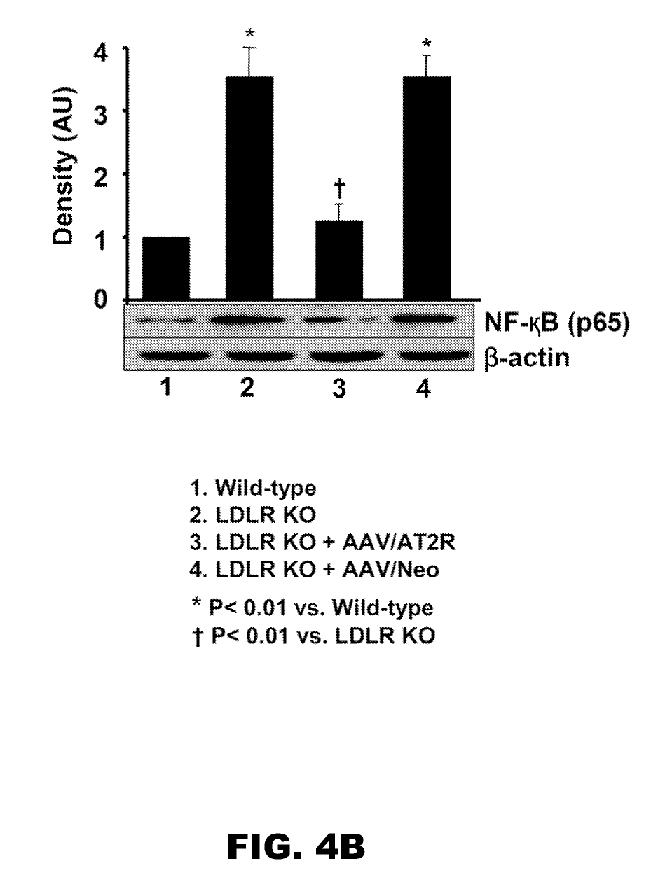

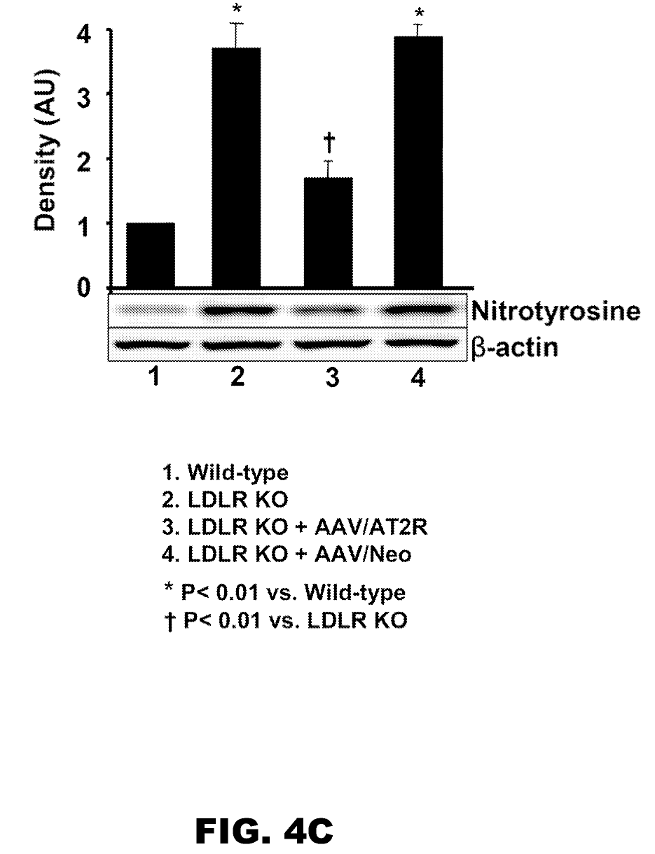

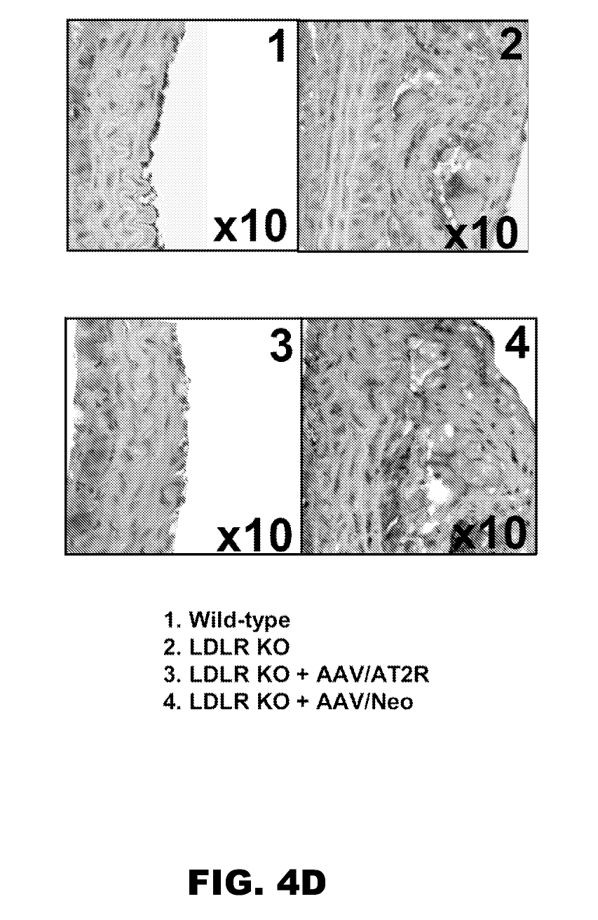

To determine the potential mechanisms of the AT2R over-expression in atherogenesis, the levels of NADPH oxidase subunits p47^{phox} and p22^{phox} and the redox-sensitive transcription factor NF-κB were examined by Western blot analysis. Both p47^{phox} and p22^{phox} subunits of NADPH oxidase were markedly increased in saline-treated and AAV/Neo-treated LDLR KO mice (vs. wild-type mice) (FIG. 4A). The upregulation of both subunits of NADPH oxidase was reduced by AT2R over-expression. Levels of NF-κB protein were also higher in saline-treated and AAV/Neo-treated LDLR KO mice (vs. the wild-type mice), and over-expression of AT2R limited the increase in NF-κB expression (FIG. 4B). To confirm the increased production of reactive oxygen species (ROS) in aorta, the presence of nitrotyrosine as an indirect marker of oxidative stress was also examined by both Western blot analysis and immunohistochemistry. Expression of nitrotyrosine was found to be higher in saline-treated and AAV/Neo-treated LDLR KO mice (vs. the wild-type mice), and over-expression of AT2R limited the increase in nitrotyrosine expression (FIGS. 4C and 4D).

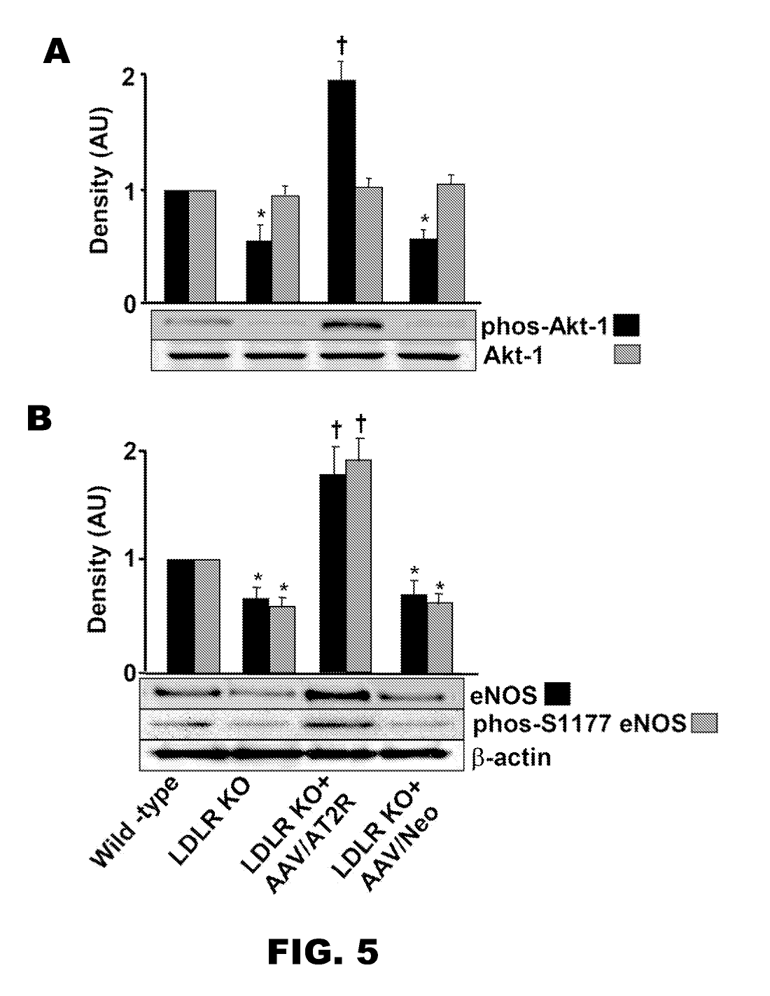

Next, the expression of Akt-1 protein and its phosphorylation in the aortic tissues were examined. Although Akt-1 protein expression was similar in all mice, Akt-1 phosphorylation was reduced in the LDLR KO mice given saline or AAV Neo (vs. wild-type mice). AT2R overexpression, however, markedly increased Akt-1 phosphorylation (FIG. 5A). Akt-1 activation regulates the expression of eNOS and its activity. Thus, the levels of eNOS protein and its phosphorylation (at S1177) were examined. As shown in FIG. 5B, the expression of eNOS protein as well as its phosphorylation was reduced in the saline-treated and AAV/Neo-treated LDLR KO mice, as compared to the wild-type mice. On the other hand, over-expression of AT2R enhanced eNOS protein expression and its phosphorylation.

The levels of HO-1, another vasodilator species that is relevant in atherogenesis, were also examined. As shown in FIG. 5C, HO-1 protein was reduced in the aortas from saline-treated and AAV/Neo-treated LDLR KO mice (vs. wild-type mice). On the other hand, delivery of AAV/AT2R enhanced its expression. The levels of LOX-1 protein were also markedly increased in the aortas from saline-treated and AAV/Neo-treated LDLR KO mice (vs. wild-type). In contrast, over-expression of AT2R reduced the increase LOX-1 protein expression (FIG. 5D).

Thus, it appears that AT2R over-expression modulates NADPH oxidase and downstream signals that may play a role in the inhibition of atherogenesis. More specifically, over-expression of AT2R may inhibits atherosclerotic lesion formation via anti-oxidant mechanisms that reduce LOX-1 expression and promote eNOS and HO-1 expression.

### Examples 4-7

### Over-Expression of AT2R Decreases Collagen Accumulation in Atherosclerotic Plaques

### Background.

Atherosclerotic regions are characterized by lipid accumulation, especially oxidized low-density lipoproteins (ox-LDL), as well as the presence of fibrous elements in different layers of the arterial wall. Examples 1-3 demonstrated the successful systemic delivery of AT2R into low-density lipoprotein receptor knockout (LDLR KO) mice with adeno-associated virus (AAV) type 2 vector, and the discovery that overexpression of AT2R reduces atherogenesis. These findings collectively suggest that AT2R activation may modulate collagen accumulation in association with atherogenesis. The following studies were designed to determine whether AT2R over-expression 1) affected collagen deposition; 2) altered the expression of osteopontin and fibronectin; 3) modulated the expression and activity of matrix metalloproteinases (MMPs); and 4) affected oxidative stress and pro-inflammatory signals involved in atherosclerotic lesion formation.

### Experimental Protocols for Examples 4-7.

The construction of the AAV/AT2R vector, generation of rAAV stocks, and animal protocols were essentially identical to those described above in Examples 1-3.

Quantitative analysis of collagen positive area. Entire aortas from the aortic arch above the aortic valves to the iliac bifurcation were harvested and embedded in paraffin. 5 μm-thick cross-sections were made at 5 pre-defined points (proximal ascending aorta, aortic arch, descending aorta, mid thoracic aorta and abdominal aorta above the renal arteries) as described by Mehta et al (2007). The sections were stained with Masson's trichrome and Picro-sirius red. The images were captured by digital imaging system and analyzed with Image pro software (Media Cybernetics, Silver Spring, Md.). Presence of area positive for collagen was recorded for each section and averaged for each mouse and expressed as ratio of entire vessel wall area. Data were obtained in 5 mice in each group.

Immunohistochemical staining. Sections of aortas (5 μm-thick) made at 5 pre-defined points as described above were incubated with primary antibodies to fibronectin or osteopontin (dilution 1:200; Santa Cruz Biotechnology Inc, Santa Cruz, Calif.) for 2 h at room temperature, rinsed with PBS, and incubated with appropriate biotinylated secondary antibodies for 30 min. The sections were then incubated with avidin-biotin complex for 30 min followed by rinse with PBS, then incubated in diaminobenzidine, and finally washed in distilled water and counterstained with hematoxylin.

Enzyme assays. Activity of MMP-2 and MMP-9 in aortic tissues was determined by zymography as described by Bruemmer et al. 2003, J. Clin. Invest. 112:1318-1331. Superoxide dismutase (SOD) activity was measured as described by Paoletti et al., 1988, Cancer Res. 48:6674-6677. For this, mouse aortic homogenates were cleared by centrifugation at 15,000×g for 30 min at 4° C., and the supernatants were used for measurements of Cu/Zn SOD activity.

Protein preparation and Western blot analysis. Aortic protein was extracted for expression analysis of pro-collagen I, osteopontin, fibronectin, MMP-2, MMP-9, CD-68, p38 mitogen-activated protein kinase (MAPK), phosphorylated p38 MAPK, p44/42 MAPK, phosphorylated p44/42 MAPK, and β-actin using standard methodologies of Western blot analysis (e.g., Mehta et al. 2007). Band density relative to β-actin was analyzed. Data were analyzed as described above for Examples 1-3.

### Example 4

### Over-Expression of AT2R and Collagen Deposition

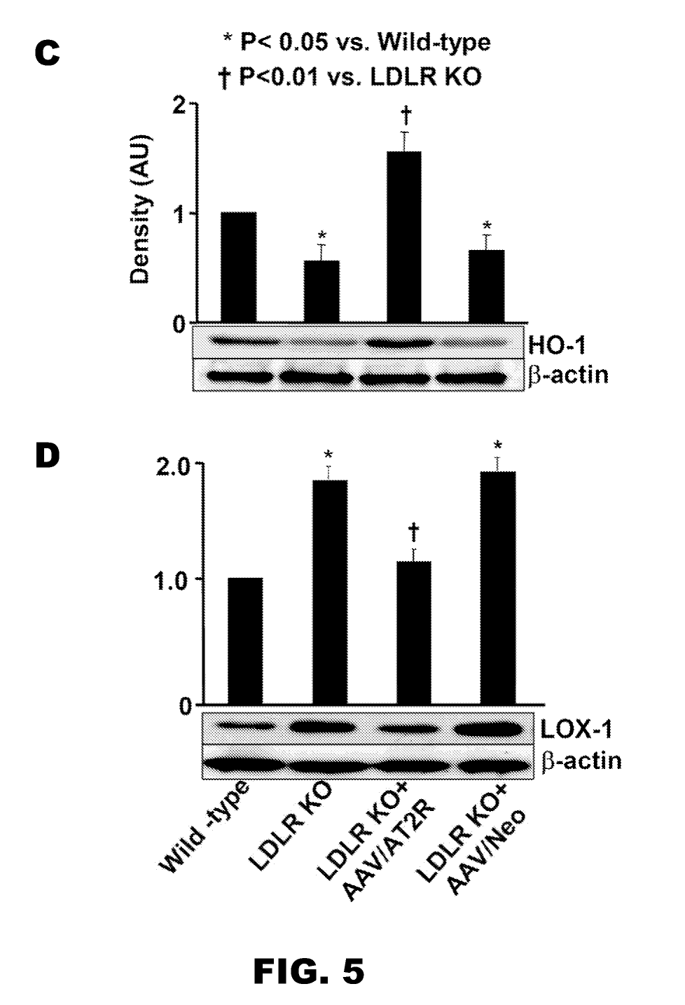

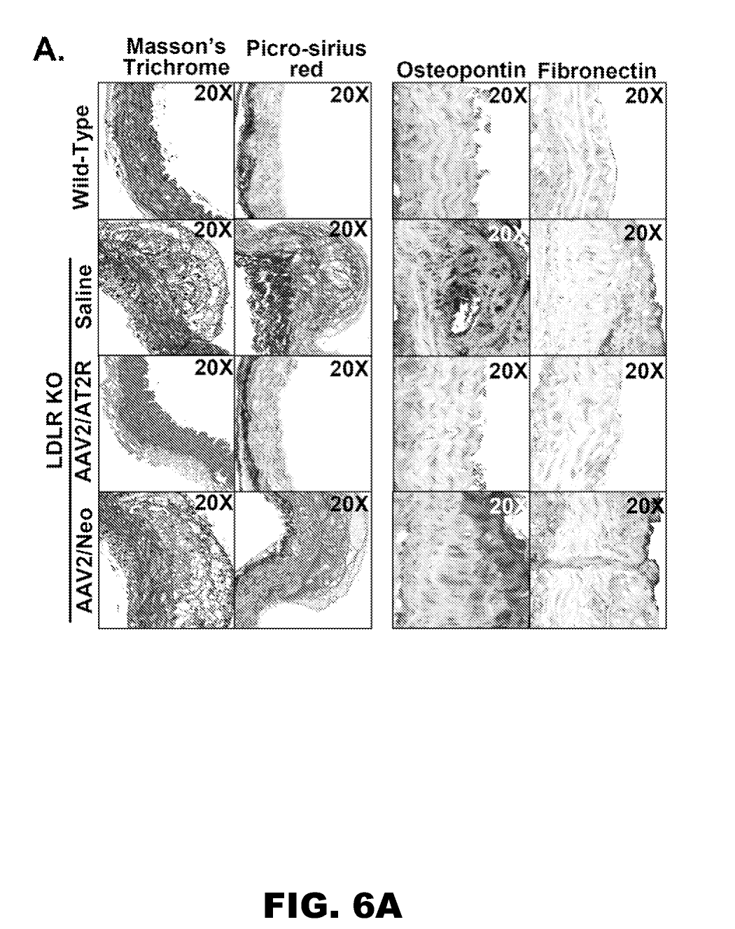

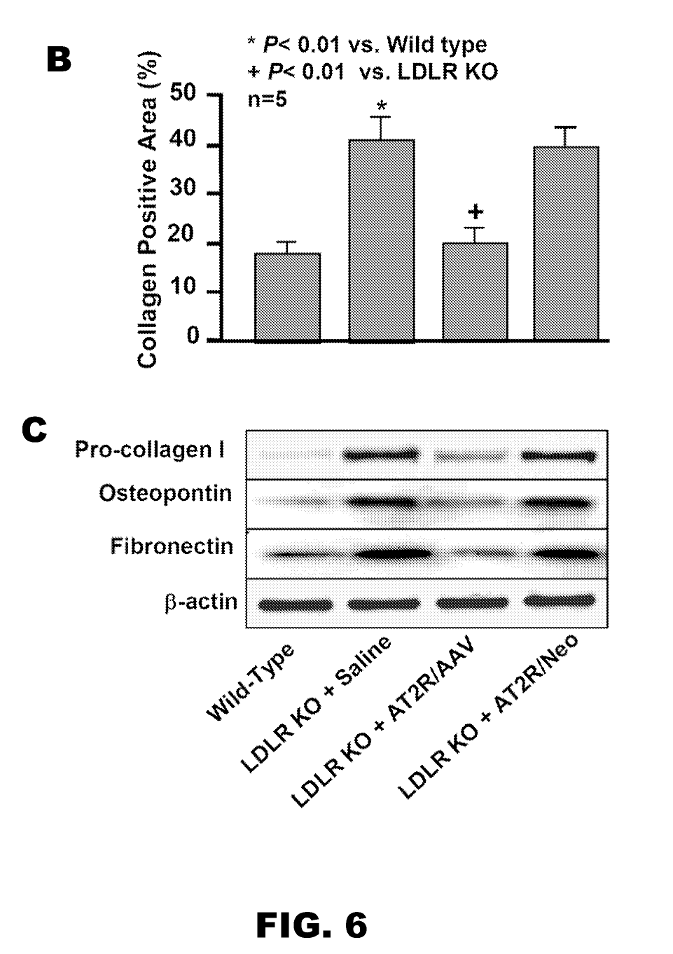

Collagen was visualized by staining aortic sections with Masson trichrome or Picro-sirius red. Representative examples of aortic sections are shown in FIG. 6A, and the Masson trichrome staining data are summarized in FIG. 6B. There was extensive collagen deposition in the aortas of LDLR KO mice given saline or AAV/Neo, encompassing 30%-45% of the aortic cross-sectional area. In contrast, collagen deposition covered only 15%-25% of the aortic wall cross-sectional area in the LDLR KO mice over-expressing AT2R. It is noteworthy that the wild-type control mice also showed some collagen positive areas, perhaps due to the high cholesterol diet.

In support of the data on collagen accumulation, pro-collagen I expression was increased in the LDLR KO mice given saline or AAV/Neo (FIG. 6C) (P<0.01 vs. wild-type mice). Over-expression of AT2R, however, reduced the increased pro-collagen I expression in the LDLR KO mice.

### Example 5

### AT2R Over-Expression and Expression of Osteopontin and Fibronectin

The levels of osteopontin and fibronectin were increased in the LDLR KO mice given saline or AAV/Neo (P<0.01 vs. wild-type mice) (see FIG. 6C). In contrast, the increased expression of osteopontin and fibronectin was reversed by over-expression of AT2R. Immunohistochemical staining of aortic sections also revealed increased levels of osteopontin and fibronectin in control LDLR KO mice, whereas the increased levels of osteopontin and fibronectin were reduced in LDLR KO mice over-expressing AT2R (FIG. 6A).

### Example 6

### AT2R Over-Expression and MMP Levels and Activity

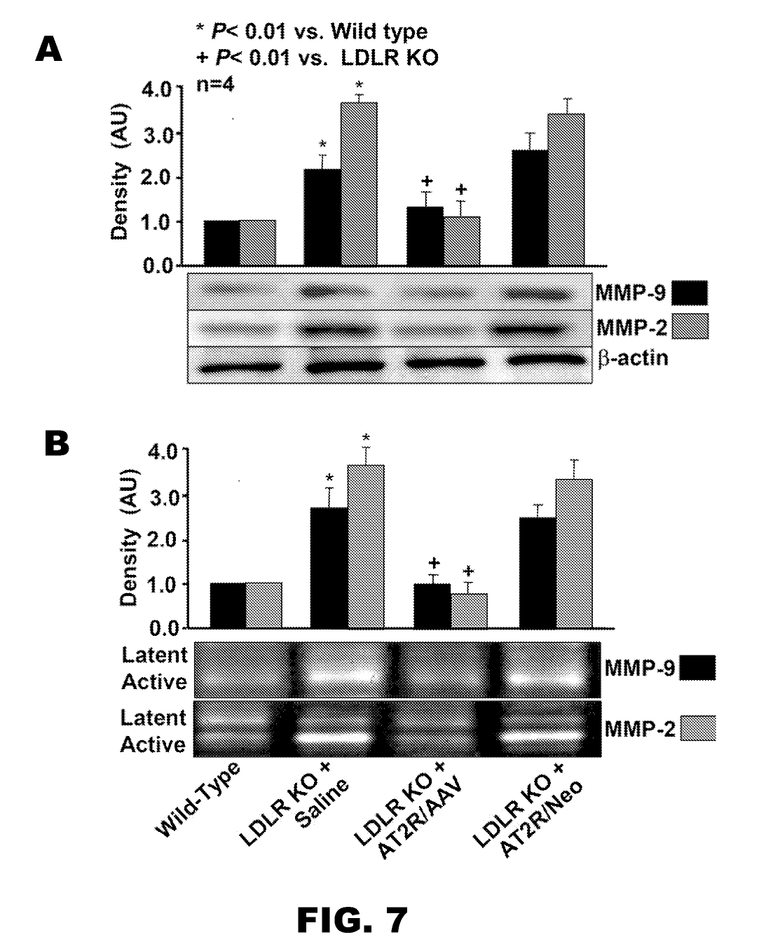

The expression of MMP-2 and MMP-9 was increased in LDLR KO given saline or AAV/Neo (P<0.01 vs. wild-type mice), whereas AT2R over-expression “normalized” the expression of MMP-2 and MMP-9 (P<0.01 vs. LDLR KO mice; P<0.05 vs. wild-type mice). See FIG. 7A.

In concert with MMP protein expression, gelatinolytic activity, which corresponds to MMP-2 and MMP-9 activity, was increased in the saline-treated LDLR KO mice or AAV/Neo-treated LDLR KO mice (FIG. 7B) (P<0.01 vs. wild-type mice). In contrast, the increased MMP activity was blocked in LDLR KO mice overexpressing AT2R (P<0.01 vs. LDLR KO mice given saline).

### Example 7

### AT2R Over-Expression and Oxidant Signaling

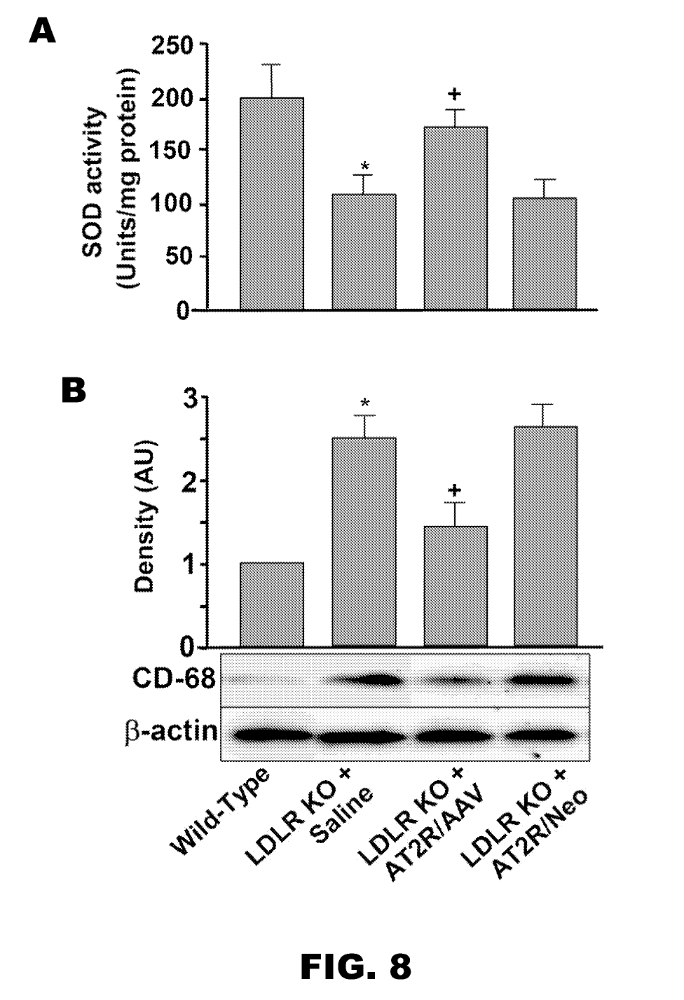

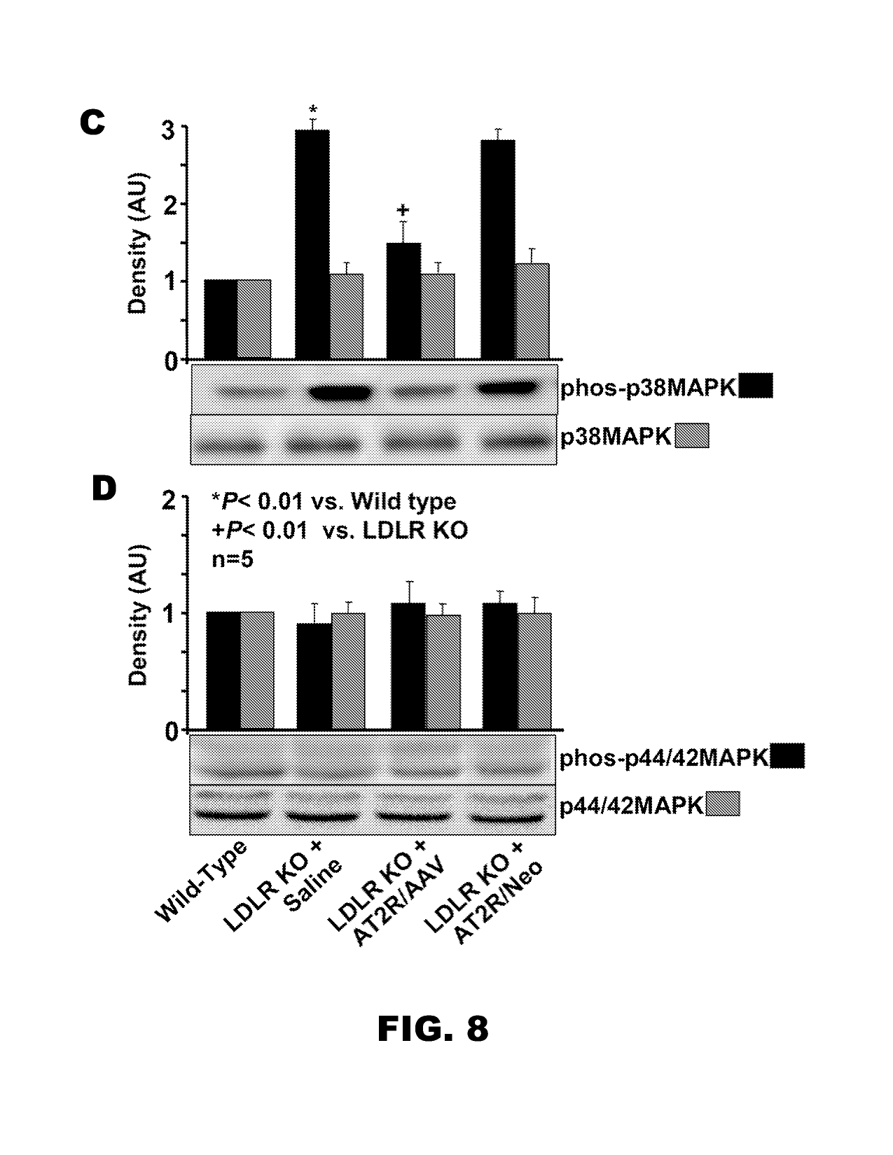

The activity of Cu/Zn SOD was decreased in the saline- or AAV/Neo-treated LDLR KO mice (P<0.01 vs. wild-type mice), whereas SOD activity in LDLR KO mice over-expressing AT2R was increased, despite the high cholesterol diet (FIG. 8A).

The expression of CD-68, a general marker of macrophage infiltration, was increased in the saline- or AAV/neo-treated LDLR KO mice (P<0.01 vs. wild-type mice). In contrast, the increased expression of CD-68 was reversed in the LDLR KO mice with upregulated AT2R (P<0.01 vs. LDLR KO) (see FIG. 8B).

The protein levels of the oxidative stress-sensitive MAPKs, both 38 and p44/42 isoforms, did not differ among the different treatment groups (FIGS. 8C and 8D). The phosphorylation of p38 MAPK, however, was increased in the LDLR KO mice given saline or AAV/Neo (P<0.01 vs. wild-type mice), but not AT2R over-expressing LDLR KO mice (FIG. 8C). The phosphorylation of p44/42 MAPK was similar in all the treatment groups (FIG. 8D).

Collectively, these data reveal that over-expression of AT2R in LDLR KO mice reduced collagen deposition in atherosclerotic regions, and this reduction in collagen deposition was associated with a reduction in associated oxidant signaling.

What is claimed is:

1. A method for treating a cardiovascular condition in a subject, the method comprising administering to the subject at least one agent that increases the level and/or activity of an angiotensin II type 2 receptor.

2. The method of claim 1, wherein the agent is selected from the group consisting of a nucleic acid encoding an angiotensin II type 2 receptor polypeptide, an angiotensin II type 2 receptor polypeptide, and an angiotensin II type 2 receptor agonist.

3. The method of claim 2, wherein the nucleic acid encoding the angiotensin II type 2 receptor polypeptide is selected from the group consisting of genomic DNA, cDNA, and RNA.

4. The method of claim 3, wherein the nucleic acid further comprises a vector selected from the group consisting of an adenovirus vector, an adeno-associated virus vector, a lentivirus vector, and a retrovirus vector.

5. The method of claim 4, wherein the adeno-associated virus (AAV) vector is selected from the group consisting of an AAV type 2 based vector, an AAV type 6 based vector, an AAV type 8 based vector, and an AAV type 9 based vector.

6. The method of claim 3, wherein the nucleic acid encoding the angiotensin II type 2 receptor polypeptide is operably linked to a promoter selected from the group consisting of a cardio-specific promoter, a vascular smooth muscle cell-specific promoter, and a fibroblast-specific promoter.

7. The method of claim 2, wherein the angiotensin II type 2 receptor agonist is a selective agonist selected from the group consisting of CPG 42112A, p-aminophenylalanine-angiotensin II, and angiotensin(1-7) heptapeptide.

8. The method of claim 1, wherein the cardiovascular condition is selected from the group consisting of atherosclerosis, coronary occlusion, myocardial infarction, renal ischemia, cerebrovascular ischemia, ischemic/reperfusion injury, hypertension, arterial aneurysm, and peripheral vascular disease.

9. The method of claim 1, wherein the agent is administered to the subject by a route selected from the group consisting of oral, inhalation, transdermal, transmucosal, intravenous, intramuscular, and subcutaneous.

10. The method of claim 1, wherein the subject is selected from the group consisting of a mammal, a human, and a companion animal.

11. The method of claim 1, wherein the agent comprises an adeno-associated virus vector comprising DNA operably linked to a promoter selected from the group consisting of a cardio-specific promoter, a vascular smooth muscle cell-specific promoter, and a fibroblast-specific promoter, and the agent is administered intravenously.

12. A method for evaluating the risk of a subject for a cardiovascular condition, the method comprising determining the ratio of angiotensin II type 2 receptor expression to angiotensin II type 1 receptor expression in a sample from the subject, wherein the risk of having or developing a cardiovascular condition decreases as the ratio increases.

13. The method of claim 12, wherein the cardiovascular condition is selected from the group consisting of atherosclerosis, coronary occlusion, myocardial infarction, renal ischemia, cerebrovascular ischemia, ischemic/reperfusion injury, hypertension, arterial aneurysm, and peripheral vascular disease.

14. The method of claim 12, wherein the sample is selected from the group consisting of blood, plasma, serum, saliva, tears, urine, and a tissue specimen.

15. The method of claim 14, wherein the tissue specimen is an arterial biopsy sample.

16. The method of claim 12, wherein the expression is measured using a method selected from the group consisting of reverse transcriptase PCR, reverse transcriptase quantitative PCR, Northern blot analysis, in situ hybridization, ELISA, Western blot analysis, immunohistochemical localization, and dot blot assay.

17. The method of claim 12, wherein the subject is selected from the group consisting of a mammal, a human, and a companion animal.

18. A combination comprising a first agent that increases the level and/or activity of an angiotensin II type 2 receptor and a second agent selected from the group consisting of an agent that decreases the level and/or activity of an angiotensin II type 1 receptor and an angiotensin-converting enzyme (ACE) inhibitor.

19. The combination of claim 18, wherein the first agent is selected from the group consisting of a nucleic acid encoding an angiotensin II type 2 receptor polypeptide, an angiotensin II type 2 receptor polypeptide, and an angiotensin II type 2 receptor agonist.

20. The combination of claim 19, wherein the nucleic acid encoding the angiotensin II type 2 receptor polypeptide is selected from the group consisting of genomic DNA, cDNA, and RNA.

21. The combination of claim 20, wherein the nucleic acid further comprises a vector selected from the group consisting of an adenovirus vector, an adeno-associated virus vector, a lentivirus vector, and a retrovirus vector.

22. The combination of claim 21, wherein the adeno-associated virus (AAV) vector is selected from the group consisting of an AAV type 2 based vector, an AAV type 6 based vector, an AAV type 8 based vector, and an AAV type 9 based vector.

23. The combination of claim 20, wherein the nucleic acid encoding the angiotensin II type 2 receptor polypeptide is operably linked to a promoter selected from the group consisting of a cardio-specific promoter, a vascular smooth muscle cell-specific promoter, and a fibroblast-specific promoter.

24. The combination of claim 19, wherein the angiotensin II type 2 receptor agonist is a selective agonist selected from the group consisting of COG 42112A, p-aminophenylalanine-angiotensin II, and angiotensin(1-7) heptapeptide.

25. The combination of claim 18, wherein the second agent is an agent that decreases the level and/or activity of an angiotensin II type 1 receptor selected from the group consisting of an angiotensin II type 1 receptor antisense nucleic acid and an angiotensin II type 1 receptor antagonist.

26. The combination of claim 25, wherein the antisense nucleic acid is selected from the group consisting an oligodeoxynucleotide, a small RNA molecule, a short interfering RNA molecule, and a short hairpin RNA molecule.

27. The combination of claim 25, wherein the angiotensin II type 1 receptor antagonist is selected from the group consisting of candesartan, eprosartan, irbesartan, losartain, olmesartan, telmisartan, and valsartan.

28. The combination of claim 18, wherein the second agent is an angiotensin-converting enzyme (ACE) inhibitor selected from the group consisting of captopril, benazepril, enalapril, fosinopril, lisinopril, perindopril, quinapril, ramipril, and zofenopril.
